(19) United States
(12) Patent Application Publication
(10) Pub. No.: US 2022/0108540 A1
(43) Pub. Date: Apr. 7, 2022

(54) DEVICES, SYSTEMS AND METHODS FOR GENERATING AND PROVIDING IMAGE INFORMATION

(71) Applicant: DeepDense Medical Ltd., Zikhron Yaakov (IL)

(72) Inventor: Yechiel Lamash, Zikhron Yaakov (IL)

(21) Appl. No.: 17/495,557

(22) Filed: Oct. 6, 2021

Related U.S. Application Data
(60) Provisional application No. 63/088,091, filed on Oct. 6, 2020.

(51) Int. Cl. G06T 19/20; G06T 7/00; G06T 7/149; G16H 30/40

(52) U.S. Cl. CPC G06T 19/20; G06T 7/0012; G06T 2219/2021; G16H 30/40; G06T 7/149

(57) ABSTRACT

Aspects of embodiments pertain to a system for providing medical information of a patient to a user, the system comprising a memory device configured to receive medical source image data descriptive of objects internal to a patient body and which were imaged in one or more image planes using one or more medical imaging modalities; segmentation image data descriptive of a 3D virtual object model that is associated with the medical source image information such that one or more segmentation image planes of the 3D virtual object model match with one or more corresponding image planes of the imaged object; and object attribute information associated with the one or more segmentation image planes.

### CROSS-REFERENCE TO RELATED APPLICATIONS

This application claims benefit from U.S. provisional application 63/088,091, filed Oct. 6, 2020, and which is incorporated herein by reference in its entirety.

### BACKGROUND

The present disclosure relates in general to the generation and providing medical information in association with medical images.

Identification of anatomical parts in medical images and related abnormalities are an essential component in various diagnostic procedures. Today, radiologists spend significant amount of time in finding the name of an anatomical parts such a vertebra or a rib's number containing some clinical finding.

The description above is presented as a general overview of related art in this field and should not be construed as an admission that any of the information it contains constitutes prior art against the present patent application.

### BRIEF DESCRIPTION OF THE FIGURES

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The figures are listed below.

FIG. 1 shows a radiological image overlayed with a region-of-interest (ROI) symbol annotated with anomaly and anatomical information, according to some embodiment.

FIG. 2 shows an example diagram of a system for providing medical imaging information.

FIG. 3 shows an example diagram of a relationship between a third party client AI device and an image data segmentation system, according to some embodiments.

FIG. 4A shows an example 3D virtual object model in conjunction with a source image in a selected image plane, according to some embodiments.

FIG. 4B shows the example 3D virtual object model in conjunction with the source image in the selected image plane, and a deformed virtual boundary, according to some embodiments.

FIG. 4C shows the updated 3D virtual object model in conjunction with the source image in the selected image plane, according to some embodiments.

FIGS. 5A-9C show radiological images in three orthogonal planes overlaid with corresponding virtual object or model contours in various deformation configurations, displayed in segmentation editing planes obtained from 3D object models.

FIG. 10 is a flowchart of a method for performing image segmentation, according to some embodiments.

### DETAILED DESCRIPTION

Aspects of disclosed embodiments pertain to systems, devices and/or methods configured to provide medical information and to systems, devices and/or methods for generating the medical information.

Some embodiments pertain to providing object characterizing information of images of objects internal to a patient

Object characterizing information may include object attribute and/or segmentation information. The medical images may be based on what may herein be referred to as “medical source image data”.

In some examples, object attribute information may be associated with or may comprise (e.g., graphic) object segmentation information, for instance, to obtain semantic object segmentation information.

In some embodiments, responsive to actionable engagement of a user with medical images, corresponding segmentation, such as an anatomical name, and object attribute information may be provided to the user, for characterizing the object.

For example, responsive to actionably engaging the source x-ray image of a hand, the system may provide an (e.g., graphic) output indicative of a fracture location (attribute information), along with the name of the fractured bone (segmentation information).

In a further example, responsive to actionably engaging the image of a blood vessel, the system may return the name of the respective arterial branch or vein, which is another example of segmentation information.

In the discussion that follows, the term “object” may for example pertain to an anatomical part, an organ, and/or an anomaly (e.g., structural anomaly, functional anomaly, tissular anomaly, etc.) associated with an anatomical part and/or organ.

In some examples, the expression “object characterization information” may include, for example, object segmentation information or labels of medical image datasets. For example, a medical source image showing multiple vertebrae may be overlaid with colored segments to assist in distinguishing one vertebra from another. Object characterization information may further include, for example, annotations of anatomical parts and/or organs, for example, by corresponding names and/or alphanumeric designations. For example, heart chambers may be annotated with their names, and bone structure such as vertebrae and ribs may be designated with respective anatomical numberings, and/or the like.

Object characterization information may further include, for example, clinical object characterizations as object attribute information. Clinical object characterizations may be descriptive of, for example, an anomaly including, for instance, a type of anomaly (e.g., malignant or non-malignant); and/or anomaly location or region where an anomaly is identified in the patient body (e.g., by indicating the name of the anatomical part in conjunction with the associated anomaly). Indicating a region where an anomaly is located may shorten a screening time by the medical professional and/or reduce computational resources (e.g., computer processing time, processing power) required to locate an anomaly within the patient body, and (thereby) possibly reducing the probability of false positives or eliminating false-positives as a result from screening irrelevant anatomical regions.

In some examples, clinical object characterizations may further include a parameter value (e.g., measure) that is indicative of organ functionality; a functional deficit of an organ; a severity of an anomaly, and/or the like. A parameter value indicative of organ functionality may for example pertain to organ size, a mechanical characteristic (e.g., elasticity, plasticity, stiffness, etc.); perfusion; hemodynamics; contrast agent dosage uptake; water diffusion rate; organ kinematics; organ dynamics; and/or the like.

In some examples, the system may be configured to mitigate or eliminate errors and/or to improve or to optimize treatment outcome. For instance, the medical information system may provide an output relating to, for instance, treatment selection and/or prioritization information including, for example, treatment prioritization and/or resources prioritization, e.g., based on resource constraints and treatment urgency.

In some embodiments, medical treatment prioritization may be based by assigning weighted factors to a plurality of parameter values relating to resource constrains and/or treatment urgency. In some examples, at least some of the weights may be predetermined, automatically adjusted by the system or user-adjustable.

In some embodiments, the output may include a structured report that may be searchable, sortable and/or filterable according to various report display criteria. For example, different reports may be displayed based on different prioritization criteria.

It is noted that the term “display” may in some implementations not only pertain to visual display, but also to an auditory display and/or a tactile display.

In some examples, the output may prioritize one medical treatment over another for the same patient; prioritize medical treatment of one patient over treatment of another patient. Considering for example identification of a blockage, hemorrhage or aneurysm in a large or main artery in a first patient and in peripheral arteries of a second patient, treatment of the first patient may be prioritized over treatment of the second patient. In some examples, the output may prioritize recommendations based on resource allocations with respect to the use of medical devices, surgical wards, hospital beds, allocation of medical professionals to various patients, and/or the like. For example, the system may be configured to assist in identifying by a medical professional whether broken ribs are associated with flail chest or not, for example, in conjunction with related symptoms such as chest pain and shortness of breath.

In some embodiments, based on clinical correlation analysis performed by the system, a clinical output may provide information about clinical symptoms and possible causes thereof. For example, the clinical output may indicate a location of artery blockage and/or hemorrhage in conjunction with identified stroke symptoms. Combining correlation analysis between symptoms and anatomical location of blockage, hemorrhage and/or aneurysm artery associated with disfunction of the respective brains' lobe may reduce uncertainty and improve automated (e.g., software-based) and/or human decision making.

In some examples, the clinical output may be automatically generated and presented as radiological reports. In some examples, a radiological report may comprise a listing of patients, characterization of anomalies detected in the patients alongside with a corresponding images in one or more imaging planes. In the report, the one or more imaging planes may be annotated with clinical information such as a type of anomaly and its location (e.g., anatomical name).

In some embodiments, the clinical output and/or anatomical segmentation can be used for navigation during catheterization.

In some embodiments, the clinical output and/or anatomical segmentation can be used for surgery planning.

In some embodiments, the clinical output can help to define anatomical markers for follow up. For example, to describe metastasis location and/or distance thereof with respect to an anatomical location such as bifurcation, bone parts etc. Using such anatomical part can be used to differentiate between a new metastasis and an old metastasis after treatment.

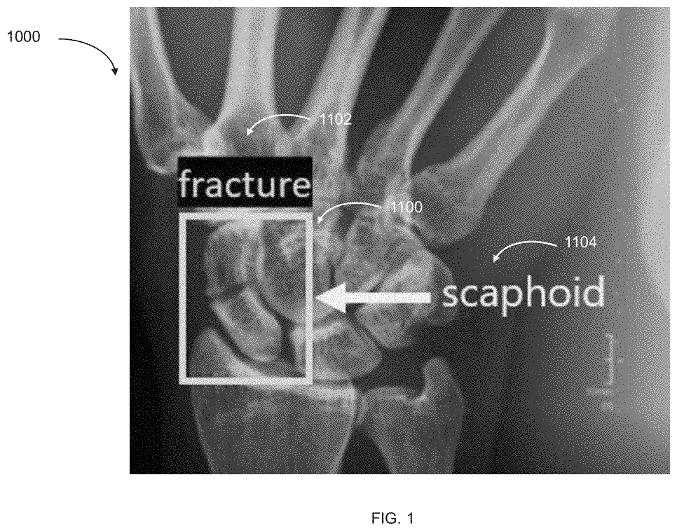

In some embodiments, part or all of clinical object characterization may be provided by a third party and combined with image characterization (e.g., segmentation) information of the system. For example, a third party vendor may provide information about an anomaly in the patient body but without indicating the anomaly's location with respect to an anatomical part. The system may complement, for instance, object attribute information indicating an area or region-of-interest (ROI) of a detected anomaly with segmentation information such as the name of an anatomical part containing the anomaly. For example, information indicating on a radiological image 1000 that a bone structure is fractured may be complemented with the name of the fractured bone. For example, an ROI 1100 of radiological image 1000 may be annotated with a clinical object characterization 1102. In the example shown in FIG. 1, a detected anomaly is annotated as “fracture. The system is configured to complement the clinical object characterization with corresponding or associated segmentation information 1104, exemplified herein as “scaphoid” characterizing the location of the anatomical anomaly identified as “fracture”.

It is noted that that ROI 1100, possible anomaly and related segmentation information may be provided in various manners. Hence, the illustration shown in FIG. 1 is non-limiting and exemplary only. For example, various shapes may be used for displaying an ROI 1100 to a user on medical images such as radiological images.

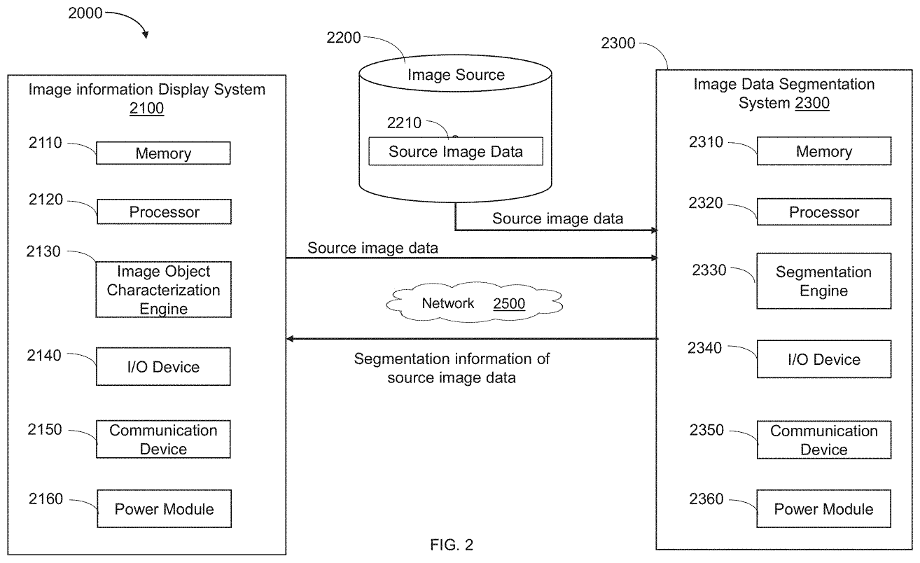

Reference is now made to FIG. 2. In some embodiments, a image data characterization (IDC) system 2000 for providing (e.g., medical) object information may be configured such that responsive to a selection made, e.g., by a user, of one or more displayed virtual objects of medical images, complementary object characterizing information such as the corresponding anatomical name of the one or more selected displayed virtual objects is returned and displayed to the user, who may be a medical professional. This feature or system configuration may not only be used for enhancing displayed radiological images by providing the latter with complementary object characterizing information, but also for training purposes of a machine learning model.

IDC system 2000 may include an Image information Display System 2100 and comprise and/or communicably cooperate with a database or source 2200 of, e.g., (medical) source images. Image source 2200 may for example comprise an electronic database and/or a medical imaging scanning source. In some examples, image source 2200 may be configured as a picture archiving and communication system (PACS).

In some examples, IDC system 2000 may further include an image data segmentation system 2300, e.g., as outlined herein below in more details.

IDC system 2000 may include a memory 2110 configured to store data and program code instructions, and a processor 2120. Processor 2120 may be configured to executed code instructions stored in memory 2110 for the processing of data, which may result in the implementation of an image object characterization (IOC) engine 2130, e.g., as outlined herein.

Image information Display system 2100 may further include an input/output (I/O) device 2140. As an output device, I/O device 2140 may display images to a user. The (e.g., medical) images displayed to the user comprises a plurality of selectable displayed or virtual objects, representing real objects internal to a patient body.

Image information Display system 2100 may further comprise at least one communication device 2150 configured to enable wired and/or wireless communication between the various components and/or modules of the system and/or apparatuses and which may communicate with each other over one or more communication buses (not shown), signal lines (not shown) and/or a network infrastructure 2500.

IOC characterization engine 2130 may be configured to associate, based on segmentation information of segmented (e.g., medical) images produced by segmentation system 2300, the selected displayed object with segmentation information. In some embodiments, the association of segmentation information with the at least one selected displayed object may be implemented through a machine learning model that was trained at segmentation system 2300 using (e.g., medical) source image data 2210, e.g., as outlined herein below in greater detail.

Source image data 2210 may be received at segmentation system 2300 from source 2200 over network 2500. Segmentation system 2300 may be employed for generating segmentation data descriptive of segmentation information that associated with at least some of the received source image data 2210. Image data having associated therewith segmentation information may herein be referred to as “segmented image data”, which may include segmented medical image data.

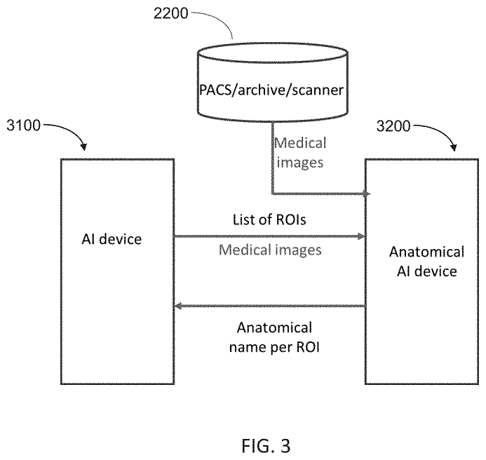

Further reference is made to FIG. 3, which is a schematic block diagram illustration of an example relationship between a third party client AI system 3100 and an anatomical AI system 3200 (also: segmentation system). Third party AI client system 3100 can send a request to get the anatomical names of one or more regions marked by anatomical AI system 3200.

It is noted that the segmentation information may be provided to display system 2100 and/or AI system 3100 in an encrypted manner.

### Segmentation Method

As mentioned herein, aspects of embodiments pertain to generating medical information, e.g., through segmentation of volumetric object data descriptive of medical images.

In some embodiments, a segmentation system may be configured to enable interactive (e.g., semi-automatic) image segmentation of an interactively deformable 3D virtual object model displayed to a user, by adjusting or adapting the 3D virtual object model to align or match or substantially match with a subject's (e.g., patient's) volumetric (e.g., medical) image data descriptive of (e.g., medical) image information displayed to the user concurrently or simultaneously with the 3D virtual object model. In some examples, a data subset descriptive of a corresponding virtual object boundary, described by a contour line, of the 3D virtual object model (e.g., a plurality of vertices of the 3D virtual object model that are descriptive of a virtual model boundary) and optional deformation of the virtual model boundary, may be displayed in overlay with a corresponding cross-sectional view of volumetric (e.g., medical) images displayed to the user. Hence, in some embodiments, vertices descriptive of such virtual model boundary are thus a subset of all vertices of the 3D virtual object model.

In one example, actionable engagement and displacement by a user of a user-selected vertex of a selected virtual contour is displayed to the user, e.g., in real-time. Once the selected vertex is moved to a desired or target position, the positions of remainder vertices of the same virtual contour are updated, e.g., at once, and displayed. The expressions “target position” and “desired position” may herein be used interchangeably.

In another example, actionable engagement and displacement by a user of a user-selected vertex of a virtual contour is displayed (e.g., in real-time) to the user, simultaneously with corresponding, e.g., real-time, displacement of the positions of remainder vertices of the same virtual contour.

In some examples, a first user input such as pressing a mouse button causes actionably engagement of a displayed vertex for displacement thereof e.g., through moving of the mouse, and release a second user input such as release of the mouse button indicates that the selected vertex is placed at the desired position. Clearly, additional or alternative input methods may be conceived such as, for example, engagements with a touch screen, use of a stylus, a gesture tracking device, and/or the like.

The virtual model contour delineates the boundary of the intersection between the 3D object model with the selected cross-section view of the volumetric image data. It is noted that merely to simplify the discussion, without be construed in a limiting manner, the expressions “contour” and “boundary” may herein be used interchangeably.

The virtual model (also known as mesh model), is implemented by vertices which may be defined by two or more matrices representing their spatial positions and relations. For example, in a matrix M comprising three columns representing x, y, and z coordinates, and a plurality of rows, each row describes the coordinates of a vertex within the 3D space. A matrix T represents the geometric relationships between neighboring vertices such that in each row, the indices of the two or more vertices represented at matrix M are stored.

Connections could be defined, for example, to generate edges, polygons defining geometric faces (e.g., triangles, rectangles, tetrahedra and/or the like), depending on the number of indices in each row of matrix T. Moreover, a global 2D (stiffness) matrix K describes mechanical relationship between vertices of the 3D model. The mechanical properties described in matrix K together with other regularization matrices such as Laplacian controls the deformation against external forces.

In some examples, the plurality of vertices descriptive of the virtual model boundary that lies within the displayed cross-section view, or may be considered to be in vicinity or close proximity of the cross-sectional view to convey the user the impression that the vertices representing the virtual model boundary lie within the plane of the cross-sectional view of the displayed medical images.

Interactive deformation of a 3D virtual object model onto patient's data in real time can be challenging. One of the challenges is related to the fact that real time (e.g., elastic) deformation of a complex object with thousands of vertices while rendering 3D volumetric imaging information is a computationally intensive process. It is noted that in some implementations, the 3D virtual object model may include several compartments to facilitate segmenting various displayed source objects displayed to the user. For example, the heart may include the four chambers of the left and right ventricles and atriums. The left ventricle may further be divided into the myocardium and blood pool etc.

As discussed herein, embodiments of disclosed systems and methods provide the user with an intuitive real time sense of 3D deformation of the 3D virtual object model for adjustment with a 3D image volume, e.g., for image segmentation purposes.

Accordingly, aspects of embodiments may also pertain to a segmentation system of image information for allowing efficient segmentation of many displayed objects having complex 3D geometry. The segmentation system allows for instance efficient segmentation of many displayed anatomical and/or organ parts such as bone structure and blood vessels for later identification.

In some embodiments, segmentation data created with the segmentation system may be used as input data for training a machine learning model. The segmentation system thus facilitates generation of large per-voxel annotated training sets for promoting artificial intelligence systems in a variety of medical imaging applications.

The segmentation system may be configured to receive medical source image data descriptive of objects which are internal to a patient body. The objects may be imaged in one or more imaging planes using one or more imaging modalities. Examples of imaging modalities may for example be based on X-ray (including, e.g., computer-tomography) based imaging technique, nuclear imaging techniques, MRI imaging techniques and/or ultrasound imaging techniques.

In some examples, medical source image data may be provided by a third party client such as, for example, a radiologist, a hospital, a health maintenance operator, another system operable to automatically detect anomalies in a radiological images, and/or the like. In some examples, source image data may also be used as input image data for training a machine learning model.

The segmentation system may further be configured to receive a 3D virtual object (image) model (e.g., a mesh model) that is representable in a plurality of segmentation editing planes.

The 3D segmentation image model may incorporate a physical model of non-rigid (e.g., elastic, elastoplastic, plastic) deformation properties of one or more virtual objects internal to a virtual patient body. In some examples, the non-rigid deformation properties may be based on a finite element elastic deformation model. In some examples, the physical model of non-rigid deformation properties may be implemented as a biomechanical elastic deformation model. In some examples, a non-rigid deformation model may have different parameter values to obtain different properties in different deformation directions. In some examples, different objects and/or object parts may be associated with different elastic deformation models or with different parameter values with respect to the same model. Considering for example a heart model, myocardial elements and blood pool elements may be associated with parameter values representing different elastic properties.

In some embodiments, implementing a physical model of non-rigid deformation properties in a 3D virtual object model may have the effect that (virtual) deformation in one plane causes corresponding (but not necessarily equal) deformation of the 3D virtual object model in other planes.

Deformation of the 3D virtual object model in one selected plane, responsive to user-interaction, may be processed and displayed to the user real-time or substantially in real-time. However, processing of corresponding deformation of the remainder of the 3D virtual object model may be computationally expensive, and thus be completed following some perceivable delay after processing and display of deformation the selected plane is already completed.

In some example implementations, the 3D mesh model may be constructed and visualized through a plurality of vertices and connections between two neighboring vertices. A physical deformable property may be associated to each string connecting between two vertices of the 3D mesh model. For a same 3D mesh model, a same physical deformable property may be associated to each one of the plurality of connecting strings.

Upon selection of a cross-sectional plane of the displayed object of the medical images, a corresponding virtual model contour of the 3D mesh model describing its boundary on the plane of cross-sectional view may be displayed in overlay with the displayed object. The user may interact with the displayed virtual model contour through an I/O device. For instance, the user may virtually engage with control points relating to the virtual model boundary overlaying the cross-sectional view of the object of the medical images to bring the virtual model boundary in alignment with boundaries or edges of a displayed object.

Actionable engagement of a control point for manipulating (e.g., positionally and/or orientationally) the virtual model contour to bring the virtual model contour in (e.g., substantial) alignment with boundaries or edges of a displayed object, may include for example, translation, rotation, scaling and/or non-rigid (e.g., elastic, elasto-plastic, or plastic) deformation.

In some examples, the control points represent the vertices of the 3D mesh model. It is noted that the I/O device may display for each cross-sectional view only the corresponding virtual model contour, significantly reducing the computational processing power or cost that would otherwise be required if the segmentation system was to display an external view of the 3D mesh model instead of a virtual model contour in correspondence with the plane of the object's cross-sectional view.

The deformation of the boundary by engaging the contour may be executed automatically, semi-automatically or by a user of the system. Merely to simplify the discussion that follows, without be construed in a limiting manner, deformation procedures may herein be discussed in the context of user-executed deformation.

Merely for the simplicity of the discussion that follows and without be construed in a limiting manner, examples described herein may be discussed in the context of elastic deformation.

For example, a user's engagement with the 3D virtual object model (or a 2D virtual contour thereof in a segmentation editing plane) for deformation purposes may be modeled as user-applied deformation forces that work against modeled friction-like forces. The modeled friction-like forces cause the displayed model to resist the user-applied deformation forces to retain or return to the shape of the 3D model object before it was subjected to the deformation forces.

In some embodiments, the elastic deformation properties may differ among various objects or object portions to mimic different biomechanical elastic deformation properties for anatomical objects.

For some example virtual objects, the associated segmentation image model may be a solid model while for some other example virtual objects the associated segmentation image model may be a shell model.

In some example implementations, the system may display the user a source image of the (e.g., medical) source image data via an I/O device. The I/O device may include input devices which are configured to convert, for example, human-generated signal such as physical movement, physical touch or pressure, and/or the like, into electrical signals as input data into the computing system.

Examples of such input devices include touch screens, hand gesture tracking devices, hand-held pointing devices (e.g., computer mouse, stylus) and/or the like. The I/O device may include output devices that are configured to convert electrical signals into outputs that can be sensed as output by a human, such as sound, light, and/or touch. Output devices may include, for example, display screens.

The I/O device allows the user to select a cross-sectional view of the segmentation image model such that the selected cross-sectional view displays a virtual model contour in a segmentation editing plane that corresponds to an imaging plane of the displayed source image.

The I/O device may be configured to display the virtual model contour in overlay with the displayed source image. In some embodiments, the displayed virtual model contour is transversely displaceable and deformable, in the corresponding segmentation editing planes, e.g., by an input provided at the I/O device by a user, in the segmentation editing plane for aligning the virtual model boundary with a source object surface boundary displayed by the source image in the matching source image plane.

A virtual model boundary that is in alignment with a source object surface boundary may herein also be referred to as “virtual target object contour”. It is noted that the terms “align”, “alignment” or any grammatical variations thereof shall encompass the meaning of the expressions “substantially align” and “substantial alignment”, respectively.

As noted above, deformation of the virtual model boundary to arrive at a visualization of a virtual target object boundary may be performed in correspondence with a physical (e.g., elastic) deformation model.

In some embodiments, segmentation information may be associated with the 3D object model. The segmentation information may include, for example, an anatomical class described, e.g., by a class number, and color-coded accordingly.

For example, a 3D object model having the form of a tube may be colored as blue and annotated with a class number relating to a “vein”.

As outlined herein, the segmentation system may allow the user to select from a plurality of displayed basic 3D object models (e.g., tube, sphere, etc.). In some examples, the user may associate segmentation information to a selected or at least two of the plurality of selectable 3D object models.

In some embodiments, a 3D object model may be displayed along with predefined segmentation information associated therewith.

In some embodiments, a 3D object model may be defined to have a plurality of virtual compartments. In some examples, each virtual compartment may have a predetermined basic shape.

In some embodiments, the user may select a 3D object model comprising, e.g., a predefined, plurality of virtual compartments which may, in some examples, be roughly configured in accordance with object information described by source image data (e.g., an anatomic region described by the volumetric image data).

In some examples, the user may select a 3D “heart” object model having four virtual compartments, wherein each compartment is assigned with corresponding segmentation information (e.g., an anatomical class number and, for example, corresponding anatomical name).

In some embodiments, the vertices defining the boundaries of each virtual compartment of a 3D object model may be color-colored to facilitate its alignment with displayed source image information such as, for example, images of a heart chamber displayed to the user.

In some embodiments, the user may generate a combined 3D object model by combining several basic 3d object models with each other defining compartments having different segmentation information associated therewith.

In some embodiments, the user may generate a combined 3D object model by dividing the combined 3D object model into several basic 3D object models and associate (e.g., different) segmentation information to each compartment.

In some embodiments, once the 3D virtual object model is set in the desired overlaying position/orientation and form, a segmentation process for generating a 3D segmented image is taking place. In such segmentation process, source image data descriptive of voxels lying inside a displayed virtual contour and, optionally, source voxels that are overlaid by the virtual contour, are assigned or associated with data descriptive of segmentation information of the corresponding 3D object model including for example, colors and/or object names (e.g., anatomical class names).

This segmentation process of displayed voxels of a 2D source plane may occur immediately after the user has positioned a selected vertex at a desired position, optionally simultaneously with the updating of the positions of the other voxels of the displayed virtual contour.

The computationally expensive process of associating segmentation information to remainder source voxels of the source image may occur, e.g., once the updating of the positions of the other vertices of the displayed virtual contour is completed and/or once the associating of the segmentation information to the currently displayed source voxels is completed. In some examples, associating segmentation information to remainder source voxels may occur while the user is displacing another vertex of the same or another displayed cross-sectional view to a desired position.

The real-time update of displayed vertices and process of associating segmentation information may provide the user with the feeling of a seamless and intuitive source image data segmentation process.

In some embodiments, the 3D object model may be first be aligned with a selected anatomical region and only then assigned, by the user, with data descriptive of the anatomical region, to result in the segmentation of the source image data.

In some embodiments, the I/O device may display orthogonal slices showing patient's body portion overlaid with an editable or deformable virtual model contour in the corresponding editing plane.

The user may actionably engage the contours displayed on the various editing planes to cause deformation of the virtual 2D contours in accordance with the physical model of elastic deformation. The 3D virtual object model may deform in accordance with a deformation vector applied, e.g., by the user, the 2D contour(s) in the segmentation editing plane, whereby the deformation occurs in accordance with the (e.g., elastic) mechanical deformation properties associated with the virtual 3D object model. Such deformation vector can represent a direction and magnitude of a force applied onto the control point.

In some examples, the deformation properties may correspond to a physical deformation model of the imaged volumetric object. In some examples, the deformation properties may be, on purpose, differ from a physical deformation model of the imaged volumetric object.

Additional or alternative conditions (e.g., boundary conditions, regularization conditions) may be taken into consideration to arrive at the target 3D segmentation including, for example, smoothness of the 3D segmentation deformation.

In some embodiments, voxel data defining the virtual target object contour and the voxels inside the contour may be associated with object attribute information that may include, for example, a name of an anatomical part and/or of a portion of the anatomical part; and/or a clinical information of the object. Voxels outside the virtual contour are excluded from being associated with the said anatomical part. Hence, the voxels of and inside the virtual contour may be annotated upon completion of a comparatively simple and intuitive image segmentation procedure.

In some embodiments, an ROI symbol (e.g., a rectangle) may be associated with a virtual target object contour. The ROI symbol may be displayed to include the area of the virtual target object contour.

In some embodiments, data representing a virtual target object contour that is aligned with an object contour may be used as source input data for source a machine learning model (e.g., an artificial neural network). In some examples, the machine learning model may assist in performing subsequent (e.g., medical) source image segmentation. In some examples, the machine learning model may be used for automated segmentation of (e.g., medical) source image data.

In some embodiments, the image segmentation method described herein may be complemented or employed in conjunction with one or more other image segmentation methods such as, for example, active contours using level-sets; atlas registration, and/or the like, e.g., to assist medical professionals to understand anatomy displayed by radiological images, facilitate report generation (e.g., dictation) and/or image analysis (e.g., diagnostics).

In some embodiments, a plurality of segmentation methods may be employed, e.g., iteratively, to arrive at the virtual target object contour. Image segmentation may for example be performed by repeatedly employing, in succession, two or more different segmentation methods to iteratively arrive or converge at the virtual target object contour.

The procedure of aligning a virtual model contour to arrive at a virtual target object contour, for a selected displayed source object, may herein referred to as “segmentation session”.

In some embodiments, the system may allow a user to select, during a segmentation session, from one or more segmentation methods to arrive at a virtual target object contour. In some embodiments, the system may automatically select or suggest a user to select from one or more segmentation methods.

Reverting now to FIG. 2, segmentation system 2300 comprises a memory 2310 configured to store data and program code instructions, and a processor 2320. Processor 2320 may be configured to executed code instructions stored in memory 2310 for the processing of data, which may result in the implementation of an image segmentation engine 2330, e.g., as outlined herein.

Image segmentation engine 2330 may enable performing processes, methods and/or procedures related to image segmentation, e.g., as described herein.

Data stored in memory 2310 may be descriptive of a 3D virtual object model and (e.g., medical) source images.

Segmentation system 2300 may comprise an I/O Device 2340. A user may actionably engage with vertices or control points of a 3D virtual object model displayed to a user by I/O device 2340, e.g., as outlined herein.

Segmentation system 2300 may further include a communication device 2350 for facilitating communication with external computing platforms and/or with components and/or modules of segmentation system 2300.

A memory may be implemented by various types of memories, including transactional memory and/or long-term storage memory facilities and may function as file storage, document storage, program storage, or as a working memory. The latter may for example be in the form of a static random access memory (SRAM), dynamic random access memory (DRAM), read-only memory (ROM), cache and/or flash memory. As working memory, memory 2110 and/or 2310 may, for example, include, e.g., temporally-based and/or non-temporally based instructions. As long-term memory, memory 2110 and/or 2310 may for example include a volatile or non-volatile computer storage medium, a hard disk drive, a solid state drive, a magnetic storage medium, a flash memory and/or other storage facility. A hardware memory facility may for example store a fixed information set (e.g., software code) including, but not limited to, a file, program, application, source code, object code, data, and/or the like.

The term “processor”, as used herein, may additionally or alternatively refer to a controller. Processors 2120 and/or 2320 may be implemented by various types of processor devices and/or processor architectures including, for example, embedded processors, communication processors, graphics processing unit (GPU)-accelerated computing, soft-core processors and/or general purpose processors.

Input/output devices 2140 and/or 2340 which may be configured to provide or receive any type of data or information. input/output devices 2140 and/or 2340 may include, for example, visual presentation devices or systems such as, for example, computer screen(s), head mounted display (HMD) device(s), first person view (FPV) display device(s), device interfaces (e.g., a Universal Serial Bus interface), and/or audio output device(s) such as, for example, speaker(s) and/or earphones. Input/output device 2140 and/or 2340 may be employed to access information generated by the system and/or to provide inputs including, for instance, control commands, operating parameters, queries and/or the like. For example, input/output device 2140 and/or 2340 may allow a user of segmentation system 2300 to actionably engage with one or more vertices and displace the one or more vertices overlaying source image information for segmentation of the latter.

Communication devices 2150 and/or 2350 configured to enable wired and/or wireless communication between the various components and/or modules of the system and which may communicate with each other over one or more communication buses (not shown), signal lines (not shown) and/or a network infrastructure.

In some examples, communication devices 2150 and/or 2350 may include I/O device drivers (not shown) and network interface drivers (not shown) for enabling the transmission and/or reception of data over a network 2500. A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G, 5G, 6G mobile networks, 3GPP, LTE, LTE advanced, Bluetooth® (e.g., Bluetooth smart), ZigBee™, near-field communication (NFC) and/or any other current or future communication network, standard, and/or system.

Network 2500 may be configured for using one or more communication formats, protocols and/or technologies such as, for example, to internet communication, optical or RF communication, telephony-based communication technologies and/or the like.

Image information Display system 2100 and segmentation system 2300 may further include a power module 2160 and 2360, respectively for powering the various components and/or modules and/or systems of the systems. A power module may comprise an internal power supply (e.g., a rechargeable battery) and/or an interface for allowing connection to an external power supply.

It will be appreciated that separate hardware components such as processors and/or memories may be allocated to each component and/or module of systems 2100 and/or 2300. However, for simplicity and without be construed in a limiting manner, the description and claims may refer to a single module and/or component. For example, although processor 2120 may be implemented by several processors, the following description will refer to processor 2120 as the component that conducts all the necessary processing functions of image information display system 2100. In some examples, the same component (e.g., processor and/or memory) may be employed for implementing a functionality of both systems 2100 and 2300.

Functionalities of one or more of the systems described herein may be implemented fully or partially by a multifunction mobile communication device also known as “smartphone”, a mobile or portable device, a non-mobile or non-portable device, a personal computer, sa laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a non-vehicular device, a stationary device and/or a home appliances control system.

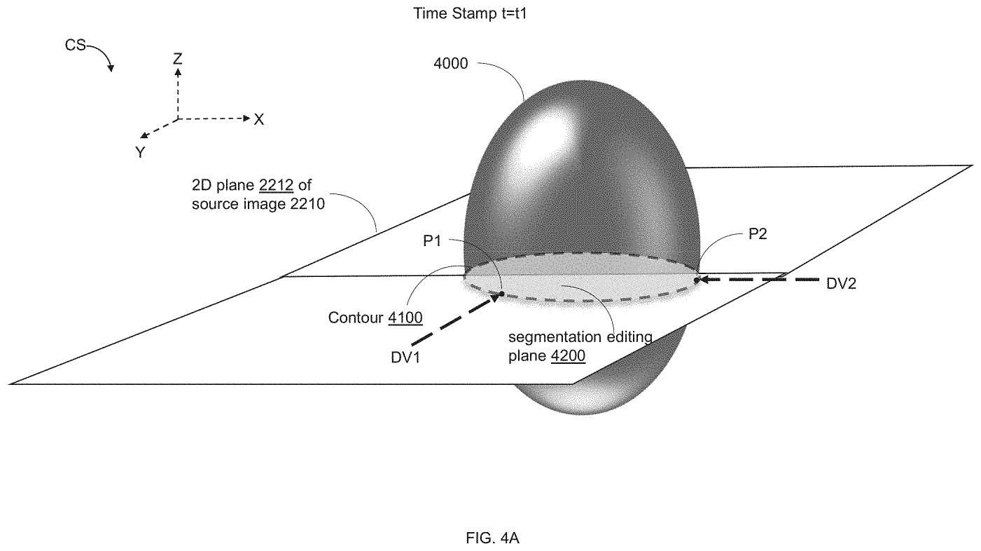

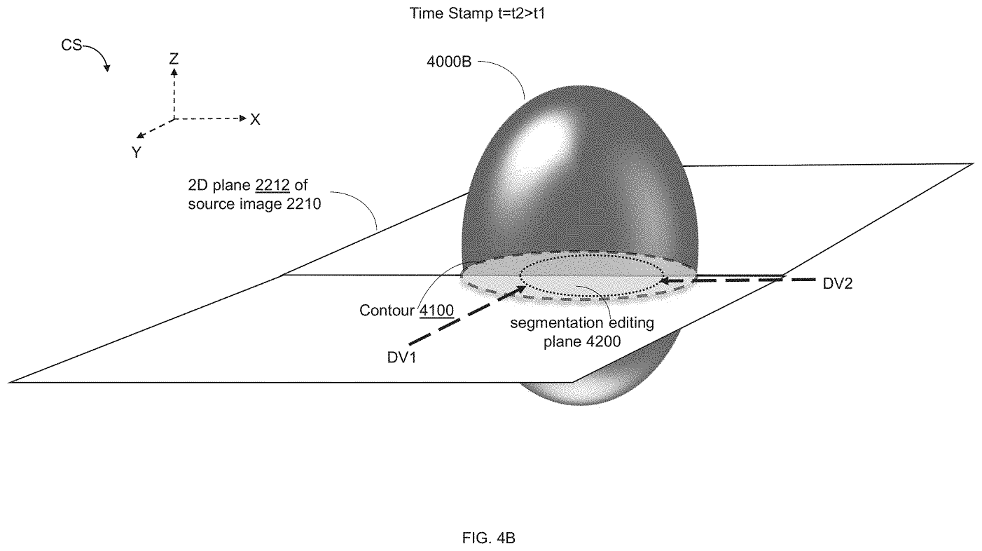

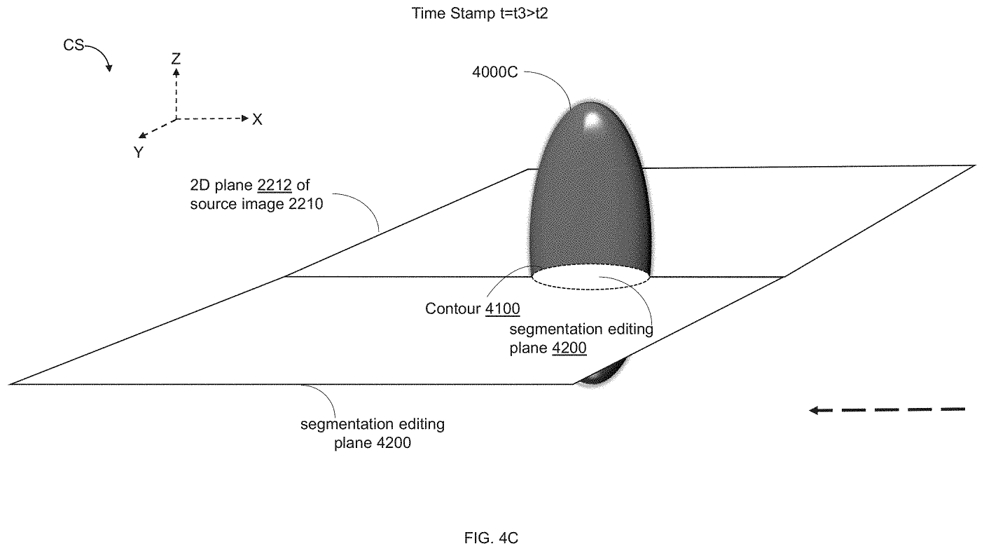

Additional reference is made to FIG. 4A, which is a schematic illustration of a deformable virtual contour 4100A defining a segmentation editing plane 4200 created by a cross-section between a 2D plane 2212 of a source (e.g., medical) image 2210 with a 3D virtual object model 4000, at time stamp t=t1, prior to actionably engaging the deformable virtual contour 4100A for causing displacement (e.g., deformation) thereof. In the example shown, segmentation editing plane 4200 is spanned unit vectors defined by x-y coordinate axes of coordinate system CS.

In some example implementations, the XY-axes may be defined to span a transverse plane, the XZ-axes may span a coronal plane, and the ZY-axes may span a sagittal plane.

Segmentation system 2300 is configured to allow a user selecting (e.g., scrolling by operably engaging with the pointer device) across different cross-sectional views, and an overlaid virtual model contour is automatically updated accordingly. Considering for instance the example shown in FIG. 4A, the user may selectively view different cross-sectional 2D planes 2212 spanned by the X-Y axes for positions selected along the Z-axis. Concurrently, for each selected 2D plane spanned by the X-Y axes, segmentation system 2300 may display in overlay the corresponding contour 4100. In addition to viewing different cross-sectional view spanned by the X-Y axes, independently from each other, segmentation system 2300 allows selecting and viewing different cross-sectional views spanned by the Z-Y axes and the Z-X axes.

Segmentation system 2300 may be configured to allow the user to seamlessly select (e.g., scroll through) various cross-sectional views represented by the volumetric image data. In some embodiments, a corresponding deformable virtual contour may be displayed in real-time in overlay for each selected cross-sectional view of the source image data.

In some examples, an initial position and/or orientation of 3D object model may randomly or pseudo-randomly selected by segmentation system 2300. In some examples, an initial position and/or orientation of 3D object model may be predetermined in segmentation system 2300. In some examples, an initial position and/or orientation of 3D object model may be user-defined.

In some examples, the shape of a 3D object model may be randomly or pseudo-randomly selected by segmentation system 2300. In some examples, a user may select a basic shape for a 3D object model from among a plurality of options presented to the user.

It is noted that the extent of a change in distance between two vertices imparted by actionably engaging a selected vertex for displacing the selected vertex relative to another vertex may result in a correspondingly proportional change to stress between to vertices, as defined by the properties of the physical model.

Deformation vectors DV1 and DV2 shown in FIG. 4A schematically illustrate a direction of force applied onto current virtual contour 4100A for causing displacement of the corresponding control points P1 and P2, to cause deformation of current virtual contour 4100 in correspondence with the physical deformation properties associated with the strings connecting between the plurality of vertices (not shown) defining 3D virtual object model 4000.

FIG. 4B shows, as a phantom, current virtual contour 4100A of FIG. 4A, illustrated by dashed line type, at time stamp t=t1 before the applied deformation, as well as virtual target or updated virtual contour 4100B, which is illustrated with dotted line type, as a result of causing displacement of vertices or control points P1 and P2 by deformation vectors DV1 and DV2, respectively. In 3D virtual object model 4000B shown in FIG. 4B, only the virtual target contour 4100B of the corresponding 3D virtual object model is shown as updated, while the remainder vertices of the 3D virtual object model 4000B remain unchanged compared to 3D virtual object model 4000A shown in FIG. 4A.

As mentioned herein, remainder vertices of 3D virtual object model 4000 that are external or that do not lie on segmentation editing plane 4200, are updated in correspondence with the magnitude and direction of the displacement imparted by the user on the control points P1 and P2, schematically illustrated in FIGS. 4A and 4B, to cause a corresponding update of 3D virtual object model 4000. FIG. 4C schematically shows updated 3D virtual object model 4000C at time stamp t=t3. The time delay between t2 to t3 reflects the time the segmentation system required to complete the computationally expensive task of updating the remainder vertices for displaying updated 3D virtual object model 4000C.

By reducing or limiting, initially, the process of displaying displacement of the subset of vertices of the segmentation editing plane, the computational processing power is significantly reduced, compared to the processing power that would be required if displacement of all vertices of the 3D virtual object model was to be processed and displayed to the user in a same processing step. In some examples, a change in displacement of the subset of vertices of the segmentation editing plane is first displayed to the user and only upon completion of displaying displacement of the subset of the vertices, the entire 3D virtual object model is updated to arrive at the updated 3D virtual object model.

For instance, a selected vertex of a segmentation editing plane may be actionably engaged and subsequently virtually displaced by the user through operably engaging and moving a pointer device (e.g., pressing a mouse button and moving the mouse). Displacement of the selected vertex may cause corresponding displacement of one or more vertices of the same segmentation editing plane, e.g., in accordance with the physical deformation properties defining a mechanical link between each two vertices. Responsive to performing a vertex disengagement operation with the pointer device (e.g., release of the mouse button), the remainder vertices of the 3D virtual object model may be updated. In some examples, due to being computationally expensive, the process of updating the remainder vertices may last a few seconds. Hence, in some embodiments, updating of the remainder vertices may not occur in real-time, whereas update of vertices of the segmentation editing may occur in real-time.

“Real-time” as used herein generally refers to the updating of information at essentially the same rate as the data is received. In the context of the present application, “real-time” can be intended to mean that a user input is acquired and processed for responsively returning an output to the user at a low enough time delay such that when the output is displayed, objects presented in the visualization move smoothly without user-noticeable judder, latency or lag. It is noted that the expressions “concurrently” and “in real-time” as used herein may also encompass the meaning of the expression “substantially concurrently” and “substantially in real-time”.

During the updating of the remainder vertices, the user may plan or engage its next step for arriving at the target model contour. This provides the user with a real time and intuitive workflow for alignment of a complex 3D mesh model onto a patient's volumetric image data.

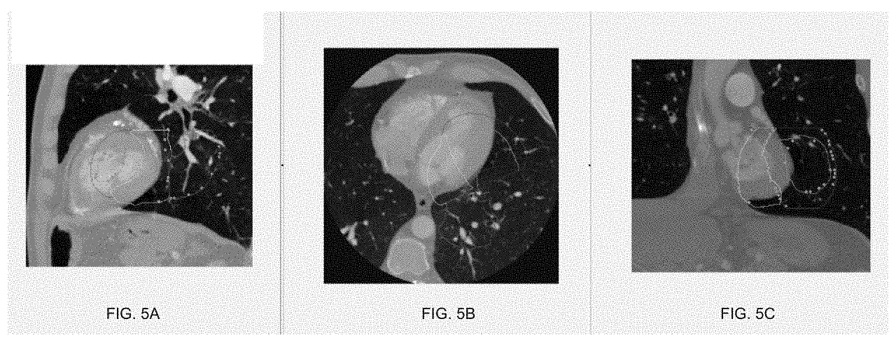

FIGS. 5A-9C show radiological example source images in three orthogonal planes overlaid with corresponding virtual object or model contours in various deformation configurations, displayed in segmentation editing planes obtained from 3D virtual object models.

For example, FIGS. 5A-C show an initial overlay of radiological example source images with virtual model contours, prior to deforming the object contours.

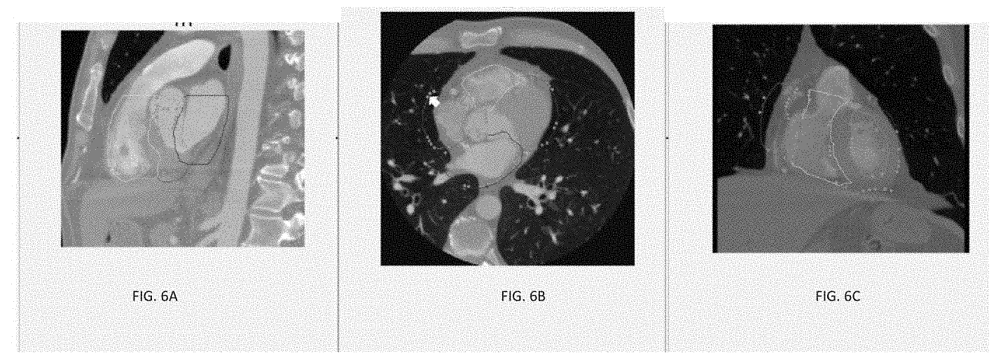

FIG. 6B shows an example of a further overlay of radiological examples source images with virtual object or model contours, and the engagement of a virtual compartment contour section (indicated by white arrow) for deformation purposes.

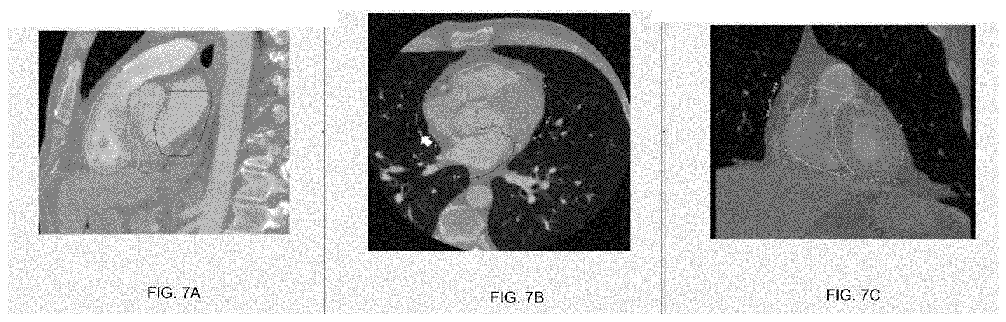

FIG. 7B shows an example of an additional overlay of radiological examples source images with virtual object or model contours after deformation was applied to the virtual compartment contour section shown in FIG. 6B, and the engagement of another virtual compartment contour section (white arrow) for deformation purposes.

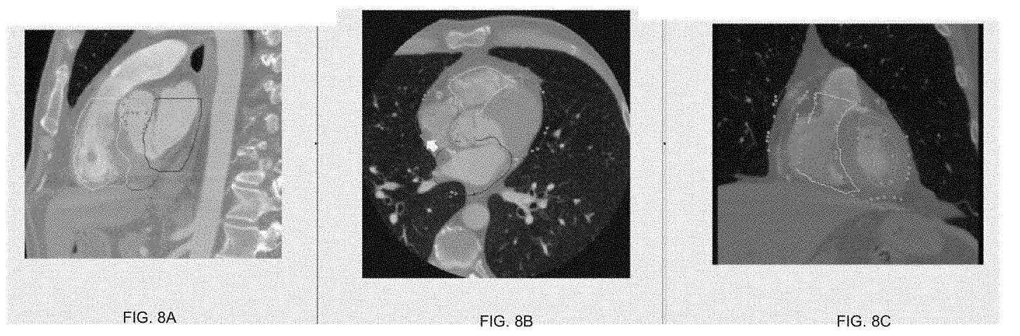

FIG. 8B shows an example of a yet additional overlay of radiological examples images with virtual object or model contours after deformation was applied to the virtual compartment contour section shown in FIG. 7B.

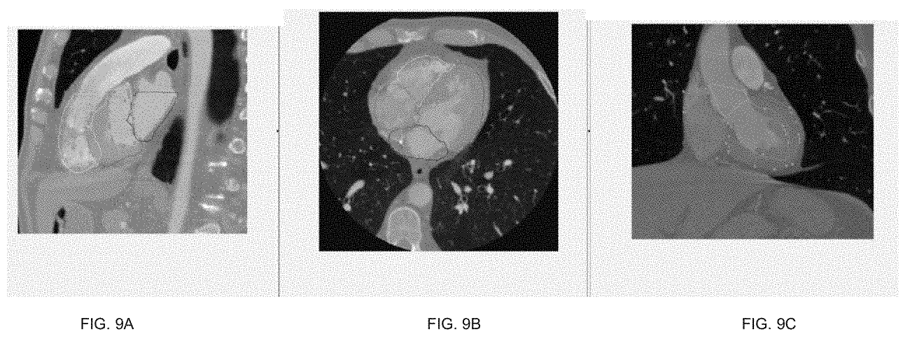

FIGS. 9A-C show the desired position of the 3D virtual object model with respect to the exemplary radiological source images.

Although embodiments described herein mainly relate to image segmentation of anatomical images, this should by no means be construed in a limiting manner. Additional applications include for example segmentation, morphing, warping and/or editing of animated videos.

### Biomechanical Model for Tissue Deformation

In some embodiments, tissue deformation can be characterized as a minimum variation of total energy, which may be described as

E=½∫_{Ω}σ^{T}εdΩ+∫_{Ω}ƒudΩ (1)

where, Ω represents the continuous domain of an elastic body, ƒ is the external force and u is the displacements in Ω. σ and ε are the stress and strain vectors, respectively.

In linear three dimensional continuum mechanics, a strain tensor is written as

[εₓₓ,ε_{yy},ε_{zz},2ε_{xy},2ε_{yz},2ε_{xz}]^{T} (2)

and stress or displacement vector is written as

[σₓₓ,σ_{yy},σ_{zz},σ_{xy},σ_{yz},σ_{xz}]^{T} (3)

The strain-stress relation is given by Hooke's law

σ=C·ε (4)

where C [6×6] is the material stiffness tensor.

Using a finite element (FE) approach, the problem domain Ω can be divided into discrete elements, with each element consisting of several nodes. The continuous displacement field is obtained through the interpolation of nodal displacements using shape functions.

The minimization of total energy in (1) using the variation principle simplifies to the following system of linear algebraic equations:

K·û=f (5)

where f is vector of applied forces, û is a vector of nodal displacements

K is the global stiffness matrix given by applying the assembly operator on the elements.

### Interactive User-Derived and Regularization Forces

Generally, the solution for the above equation requires boundary conditions that add constraints to the FE system of linear equations. Here instead, we add the identity matrix to the stiffness matrix with a weighting parameter α to inhibit the movement of the organ. In other words, the user-applied local force deforms the organ which has separate forces that keep it in its place. Such forces can be interpreted as motion resistors that work against the user-derived force.

The force to the control point is extracted from the control point's displacement applied by the user. The user displaces control points one at a time, thus, the vector of displacements û and the vector of forces ƒ contain non-zero elements only in the displaced control point indices.

The local force vector is obtained by:

ƒ=kû (6)

where, k is a predefined elastic scalar which relates between the local user displacement and the derived force.

After calculating the local force on the displaced control point, it is inserted at the appropriate indices of the global force vector, with zero values at the rest of the indices.

Other Regularization Matrices for Mesh Deformation

One option for a regularization matrix that impose smoothness on deformation of a mesh model is by applying the Laplacian or weighted Laplacian on the mesh vertices. Such formulation may or may not use the elastic properties of the deformed model. Other formulations that impose smoothness in deforming the mesh by local force or multiple local forces can also be used.

To generalize both to the stiffness matrix and Laplacian, the generalized mesh displacements can be obtained by:

û=(γL+βK+αl)⁻¹·ƒ (7)

where:

K is the elastic global stiffness matrix.

L is the Laplacian or weighted Laplacian matrix.

l is the identity matrix.

α [0, ∞) is a predefined parameter that controls mesh inhibitory forces.

β [0, ∞)is a predefined parameter that controls the stiffness matrix K.

γ [0, ∞)is a predefined parameter that controls the Laplacian matrix L.

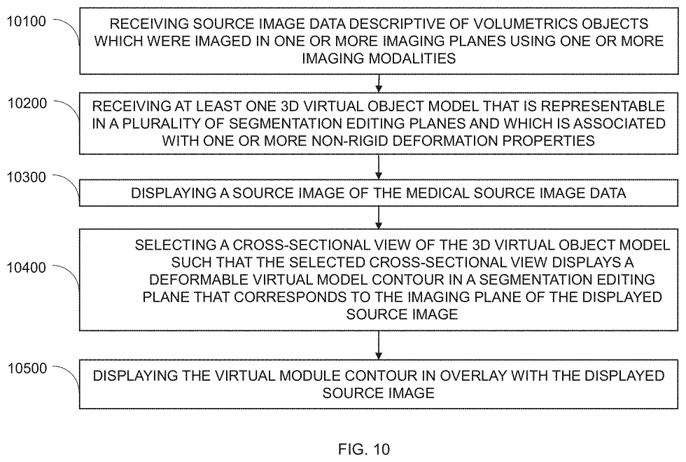

Additional reference is now made to FIG. 10. In some embodiments, a method for generating image information may include, for example, receiving source image data descriptive of volumetrics objects which were imaged in one or more imaging planes using one or more imaging modalities (block 10100).

In some embodiments, the method may further include receiving at least one 3d virtual object model that is representable in a plurality of segmentation editing planes and which is associated with one or more non-rigid deformation properties (block 10200).

In some embodiments, the method may include displaying a source image of the source image data (block 10300).

In some embodiments, the method may further include selecting a cross-sectional view of the 3d virtual object model such that the selected cross-sectional view displays a deformable virtual model contour in a segmentation editing plane that corresponds to the imaging plane of the displayed source image (block 10400).

In some embodiments, the method may include displaying the virtual module contour in overlay with the displayed source image (block 10500).

### Additional Examples

Example 1 pertains to a segmentation system, comprising: a memory device configured to receive:

source image data descriptive of volumetrics objects which were imaged in one or more imaging planes using one or more imaging modalities;

a 3D virtual object model that is representable in a plurality of segmentation editing planes and which is descriptive of non-rigid deformation properties of one or more of the imaged volumetrics objects;

a processor that is configured to perform the following:

displaying a cross-section view of a volumetric source image;

generating displaying a deformable virtual model contour in a segmentation editing plane that corresponds to the imaging plane of the displayed cross-section view of the volumetric source image; and

displaying the deformable virtual model contour in overlay with the displayed cross-section view of the volumetric source image.

Example 2 includes wherein the displayed virtual model contour is deformable to obtain a target contour that is aligned with at least one object portion displayed in the cross-section view of the volumetric source image, wherein deformation of the virtual model contour causes deformation of the (e.g., entire) 3D virtual object model in accordance with the non-rigid deformation properties associated with the at least one 3D virtual object modeland optionally the subject matter of example 1.

Example 3 includes a segmentation system is configured such that a virtual model contour is actionably engageable by a user to impart, by the user, displacement of one or more control points of the virtual model contour for changing display of a current position and/or orientation of the virtual model contours of all editing planes to an updated position and/or orientation and, optionally, the subject matter of examples 1 and/or 2.

Example 4 includes a segmentation system configured to display the virtual model contour at the updated position and/or orientation and, optionally comprises the subject matter of any one or more of the Examples 1 to 3.

Example 5 includes that a segmentation system is configured such that deformation imparted on the virtual model contour is displayed in real-time and, optionally, the examples of any one or more of the examples 1 to 4.

Example 6 includes that a virtual model contour that is represented by a subset of vertices of a plurality of vertices defining the 3D virtual object model and, optionally the subject matter of any one or more of the examples to 1 to 5.

Example 7 includes wherein positions of remainder vertices descriptive of the 3D virtual object model are updated after displaying the virtual model contour and the updated position and/or orientation and, optionally, any one or more of the examples 1 to 6.

Example 8 includes, wherein at least one of the plurality of displayed objects is internal to a virtual patient body and, optionally any one or more of the examples 1 to 7.

Example 9 includes wherein the non-rigid deformation properties pertain to one of the following: elastic deformation properties, plastic deformation properties, elasto-plastic deformation properties and, optionally any one or more of the examples 1 to 8.

Example 10 includes wherein employing the physical model of non-rigid deformation has the effect that deformation in one plane causes corresponding deformation of the 3D virtual object model in the remainder planes of the 3D virtual object model and, optionally, any one or more of the examples 1 to 9.

Example 11 includes, wherein the non-rigid deformation model comprises a biomechanical deformation model and, optionally, any one or more of the examples 1 to 10.

Example 12 includes wherein the non-rigid deformation is spatially variant or invariant and, optionally, any one or more of the examples 1 to 11.

Example 13 includes wherein data descriptive of the target contour is used as input data to train a machine learning model to facilitate e.g., automatic or semi-automatic, image segmentation of source images and, optionally any one or more of the examples 1 to 12.

Example 14 includes wherein the segmentation system is configured to associate attribute information to the target contour, wherein the object attribute information can be used, for example, for the following: treatment selection; patient prioritizations; information about symptoms and causes thereof; surgery planning, 3D printing based on surgery planning, or any combination of the above.

Example 15 includes the wherein a segmentation system is further configured to automatically identify and indicate the location of an anomaly in the medical imagery, in conjunction with the anatomical part name containing the anomaly and, optionally, any one or more of the examples 1 to

[symbol]

Example 16 pertains to a system for providing medical information of a patient to a user, the system comprising:

a memory device configured to receive:

[symbol] (e.g., medical) source image data descriptive of source objects internal to a patient body and which were imaged in one or more image planes using one or more medical imaging modalities;

segmentation image data descriptive of a segmentation image model that is associated with (e.g., medical) source image information such that one or more segmentation image planes of the segmentation image model match with one or more corresponding image planes of the imaged source object, wherein the segmentation image model is based on a deformed 3D virtual object model; and

object attribute information associated with the one or more segmentation image data.

Example 17 includes an output unit, a processor that is configured to cause the output unit to output images of objects in the one or more image planes along with corresponding object attribute information and, optionally the subject matte of any one or more of the examples 1 to 16.

Example 18 includes wherein the object attribute information further includes a name of an anatomical part and/or of a portion of the anatomical part; a clinical characterization of the object; or both and, optionally the subject matter of any one or more of examples 1 to 17.

Example 19 includes wherein the object attribute information further includes: object or organ size; a mechanical characteristic; perfusion; hemodynamics; contrast agent dosage uptake; water diffusion rate; contrast agent dosage uptake; organ kinematics and/or dynamics; any combination of the above, and optionally, the subject matter of one or more of the examples 1 to 18.

Example 20 pertains to an image data segmentation method, comprising: receiving source image data descriptive of volumetrics objects which were imaged in one or more imaging planes using one or more imaging modalities; receiving at least one 3D virtual object model that is representable in a plurality of segmentation editing planes and which is associated with non-rigid deformation properties; optionally displaying a source image of the source image data; optionally selecting a cross-sectional view of the 3D virtual object model such that the selected cross-sectional view displays a deformable virtual model contour in a segmentation editing plane that corresponds to an imaging plane of the displayed source image; and optionally displaying the deformable virtual model contour in overlay with the displayed source image.

Example 21 includes actionably engaging the virtual model contour to impart displacement of one or more control points of the virtual model contour for changing display of a current position and/or orientation of the virtual model contour to an updated position and/or orientation; and displaying the virtual model contour at the updated position and/or orientation, and, optionally, the subject matter of any one or more of the examples 1 to 20.

Example 22 includes wherein displaying of an object virtual model contour is executed in real-time and, optionally, the subject matter of any one or more of the examples 1 to 21.

Example 23 pertains to a system for providing (e.g., medical) information about a patient to a user, the system comprising: a memory device configured to receive (e.g., medical) source image data descriptive of objects internal to a patient body and which were imaged in one or more image planes using one or more medical imaging modalities; segmentation image data descriptive of a segmentation image model, obtained by positioning and/or deforming a 3D virtual object model to arrive at a target position, wherein the segmentation image model is associated with source image information such that one or more segmentation image planes match with one or more corresponding image planes of the imaged source object; and object attribute information associated with the one or more segmentation image planes.

Example 24 comprises an output unit; and a processor that is configured to cause the output unit to output images of objects in the one or more image planes along with corresponding object attribute information and, optionally, the subject matter of any one or more of Example 23.

Example 25 includes wherein the object attribute information comprises one of the following: an indication of a region-of-interest (ROI) inside the patient body; object characterization; or both and, optionally, the subject matter of any one or more of Examples 23 or 24.

Example 26 includes wherein the object characterization includes providing a name of an anatomical part and/or of a portion of the anatomical part; a clinical characterization of the object; or both and, optionally, the subject matter of any one or more of Examples 23 to 25.

Example 27 includes wherein the clinical characterization includes a location of an anomaly with respect to an anatomical part; a type of an anomaly; a measure indicative of organ functionality and/or of severity of an anomaly; or any combination of the above; and, optionally, the subject matter of any one or more of Examples 23 to 26.

Example 28 includes wherein the clinical characterization comprises an output relating to treatment selection and/or patients' prioritization and, optionally, the subject matter of any one or more of Examples 23 to 27.

Example 29 includes wherein the objective measurement includes with the respect to the object: size; a mechanical characteristic; perfusion; hemodynamics; contrast agent dosage uptake; water diffusion rate; contrast agent dosage uptake; organ kinematics and/or dynamics; any combination of the above and, optionally, the subject matter of any one or more of Examples 23 to 28.

Example 30 relates to a segmentation system of image data, the system comprising: a memory device configured to receive (e.g., medical) source image data descriptive of objects internal to a patient body which were imaged in one or more imaging planes using one or more imaging modalities; a segmentation image model that is representable in a plurality of segmentation editing planes and which is descriptive of non-rigid (e.g., elastic, elastoplastic) deformation properties of one or more virtual objects internal to a virtual patient body; and a processor that is configured to perform the following: displaying a source image of the medical source image data; selecting a cross-sectional view of the segmentation image model such that the selected cross-sectional view displays a virtual model contour in a segmentation editing plane that corresponds to an imaging plane of the displayed source image; and displaying the virtual model contour in overlay with the displayed source image.

In some examples, the displayed virtual model contour is deformable to obtain a matching or target contour that is aligned with an object displayed by the source image, wherein deformation of the virtual model contour is based on the non-rigid deformation model of the corresponding segmentation image model.

Example 40 includes wherein employing the physical model of non-rigid deformation has the effect that deformation in one plane causes corresponding deformation of the 3D virtual object model in other planes to obtain segmentation data and, optionally, the subject matter of Example 39.

Example 41 includes wherein the non-rigid deformation model includes a biomechanical elastic deformation model and, optionally, the subject matter of any one or more of Examples 39 to 40.

Example 42 includes wherein the non-rigid deformation is spatially variant or invariant optionally, the subject matter of any one or more of Examples 39 to 41.

Example 43 includes wherein the selected cross-sectional view is used as source input data to train a machine learning model to facilitate medical source image segmentation optionally, the subject matter of any one or more of Examples 39 to 42.

Example 44 includes wherein the object attribute information is used for the following: treatment selection; patient prioritizations; information about symptoms and causes thereof, e.g., to facilitate a decision making process by a human and/or by a computerized system; to implement a decision support system, or any combination of the above and, optionally, the subject matter of any one or more of Examples 39 to 43.

Example 45 includes wherein the processor is further configured to indicate the location of an anomaly in conjunction with the anatomical part name containing the anomaly, and, optionally, optionally, the subject matter of any one or more of Examples 39 to 44.

Example 46 includes wherein an output provided by the system pertains to one of the following: flail chest diagnostics; identification of blockage in large artery and small peripheral arteries for patient prioritization and decision making; indicating correlation between location of artery blockage/hemorrhage and patient's symptoms during brain stroke; or any combination of the above and, optionally, the subject matter of any one or more of Examples 39 to 45.

Example 47 includes an image data segmentation system, comprising: a memory device configured to receive:

source image data descriptive of volumetrics objects which were imaged in one or more imaging planes using one or more imaging modalities;

a 3D virtual object model that is representable in a plurality of segmentation editing planes and which is descriptive of non-rigid deformation properties of one or more of the imaged volumetrics objects;

a processor that is configured to perform the following:

displaying a source image of the source image data;

selecting a cross-sectional view such that the selected cross-sectional view displays a deformable virtual model contour in a segmentation editing plane that corresponds to the imaging plane of the displayed source image; and

displaying the deformable virtual model contour in overlay with the displayed source image, wherein the displayed virtual model contour is deformable to obtain a target contour that is aligned with at least one object displayed by the source image, wherein deformation of the virtual model contour causes deformation of the entire 3D virtual object model in accordance with the non-rigid deformation properties associated with the at least one 3D virtual object model.

It is important to note that the methods described herein and illustrated in the accompanying diagrams shall not be construed in a limiting manner. For example, methods described herein may include additional or even fewer processes or operations in comparison to what is described herein and/or illustrated in the diagrams. In addition, method steps are not necessarily limited to the chronological order as illustrated and described herein.

Any digital computer system, unit, device, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may directly loadable into an internal memory of a digital computer, comprising software code portions for performing the methods and/or processes as disclosed herein.

The methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The term “engine” may comprise one or more computer modules, wherein a module may be a self-contained hardware and/or software component that interfaces with a larger system. A module may comprise a machine or machines executable instructions. A module may be embodied by a circuit or a controller programmed to cause the system to implement the method, process and/or operation as disclosed herein. For example, a module may be implemented as a hardware circuit comprising, e.g., custom VLSI circuits or gate arrays, an Application-specific integrated circuit (ASIC), off-the-shelf semiconductors such as logic chips, transistors, and/or other discrete components. A module may also be implemented in programmable hardware devices such as field programmable gate arrays, programmable array logic, programmable logic devices and/or the like.

The term “random” also encompasses the meaning of the term “substantially randomly” or “pseudo-randomly”.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms “substantially”, “'about” and/or “close” with respect to a magnitude or a numerical value may imply to be within an inclusive range of −10% to +10% of the respective magnitude or value.

“Coupled with” can mean indirectly or directly “coupled with”.

It is important to note that the method may include is not limited to those diagrams or to the corresponding descriptions. For example, the method may include additional or even fewer processes or operations in comparison to what is described in the figures. In addition, embodiments of the method are not necessarily limited to the chronological order as illustrated and described herein.

Discussions herein utilizing terms such as, for example, “processing”, “computing”, “calculating”, “determining”, “establishing”, “analyzing”, “checking”, “estimating”, “deriving”, “selecting”, “inferring” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes. The term determining may, where applicable, also refer to “heuristically determining”.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

Terms used in the singular shall also include the plural, except where expressly otherwise stated or where the context otherwise requires.

In the description and claims of the present application, each of the verbs, “comprise” “include” and “have”, and conjugates thereof, are used to indicate that the data portion or data portions of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made. Further, the use of the expression “and/or” may be used interchangeably with the expressions “at least one of the following”, “any one of the following” or “one or more of the following”, followed by a listing of the various options.

As used herein, the phrase “A,B,C, or any combination of the aforesaid” should be interpreted as meaning all of the following: (i) A or B or C or any combination of A, B, and C, (ii) at least one of A, B, and C; (iii) A, and/or B and/or C, and (iv) A, B and/or C. Where appropriate, the phrase A, B and/or C can be interpreted as meaning A, B or C. The phrase A, B or C should be interpreted as meaning “selected from the group consisting of A, B and C”. This concept is illustrated for three elements (i.e., A,B,C), but extends to fewer and greater numbers of elements (e.g., A, B, C, D, etc.).

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or optional implementation are not to be considered essential features of those embodiments, unless the embodiment, example and/or optional implementation is inoperative without those elements.

It is noted that the terms “in some embodiments”, “according to some embodiments”, “for example”, “e.g.”, “for instance” and “optionally” may herein be used interchangeably.

The number of elements shown in the Figures should by no means be construed as limiting and is for illustrative purposes only.

It is noted that the terms “operable to” can encompass the meaning of the term “modified or configured to”. In other words, a machine “operable to” perform a task can in some embodiments, embrace a mere capability (e.g., “modified”) to perform the function and, in some other embodiments, a machine that is actually made (e.g., “configured”) to perform the function.

Throughout this application, various embodiments may be presented in and/or relate to a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

While the invention has been described with respect to a limited number of embodiments, these should not be construed as limitations on the scope of the invention, but rather as exemplifications of some of the embodiments.

What is claimed is:

1. An image data segmentation system, comprising:
a memory device configured to receive:
source image data descriptive of volumetrics objects which were imaged in one or more imaging planes using one or more imaging modalities;
a 3D virtual object model that is representable in a plurality of segmentation editing planes and which is descriptive of non-rigid deformation properties of one or more of the imaged volumetrics objects;
a processor that is configured to perform the following:
displaying a cross-section view of a volumetric source image; and
generating a deformable virtual model contour in a segmentation editing plane that corresponds to the imaging plane of the displayed cross-section view of the volumetric source image; and
displaying the deformable virtual model contour in overlay with the displayed cross-section view of the volumetric source image,
wherein the displayed virtual model contour is deformable to obtain a target contour that is aligned with at least one object portion displayed in the cross-section view of the volumetric source image, wherein deformation of the virtual model contour causes deformation of the 3D virtual object model in accordance with the non-rigid deformation properties associated with the at least one 3D virtual object model.

2. The segmentation system of claim 1, configured such that the virtual model contour is actionably engageable by a user to impart, by the user, displacement of one or more control points of the virtual model contour for changing display of a current position and/or orientation of the virtual model contour to an updated position and/or orientation; and
to display the virtual model contours of all editing planes at the updated position and/or orientation.

3. The segmentation system of claim 1, configured such that the deformation imparted on the virtual model contour is displayed in real-time.

4. The segmentation system of claim 1, wherein the virtual model contour is represented by a subset of vertices of a plurality of vertices defining the 3D virtual object model.

5. The segmentation system of claim 4, wherein positions of remainder vertices descriptive of the 3D virtual object model are updated after displaying the virtual model contour and the updated position and/or orientation.

6. The segmentation system of claim 1, wherein at least one of the plurality of displayed objects is internal to a virtual patient body.

7. The segmentation system of claim 1, wherein the non-rigid deformation properties pertain to one of the following: elastic deformation properties, plastic deformation properties, elasto-plastic deformation properties.

8. The segmentation system of claim 1, wherein employing the physical model of non-rigid deformation has the effect that deformation in one plane causes corresponding deformation of the .3D virtual object model in the remainder planes of the 3D virtual object model.

9. The segmentation system of claim 1, wherein the non-rigid deformation model includes:
a biomechanical deformation model descriptive of non-rigid deformation that is spatially variant or invariant.

10. The segmentation system of claim 1, wherein data descriptive of the target contour is used as training input data to train a machine learning model to facilitate automatic or semi-automatic source image segmentation.

11. The segmentation system of claim 1, further configured for associating attribute information to the target contour, wherein the object attribute information is used for the following:
treatment selection; patient prioritizations; information about symptoms and causes thereof; surgery planning, 3D printing based on surgery planning, or any combination of the above.

12. The segmentation system of claim 1, wherein the processor is further configured to automatically identify and indicate the location of an anomaly in medical source image data, in conjunction with the anatomical part name containing the anomaly.

13. The segmentation system of claim 1, wherein the 3D virtual object model may include several compartments to facilitate segmenting different source objects displayed to the user.

14. An image information display system for providing medical information of a patient to a user, the system comprising:
a memory device configured to receive:
medical source image data descriptive of objects internal to a patient body and which were imaged in one or more image planes using one or more medical imaging modalities;
segmentation image data descriptive of a segmentation image model that is associated with the medical source image information such that one or more segmentation image planes of the segmentation image model match with one or more corresponding image planes of the imaged object, wherein the segmentation image model is obtained by associating a deformable 3D virtual object model with source images; and
object attribute information associated with the one or more segmentation image planes.

15. The image information display system of claim 14, further comprising:
an output unit; and
a processor that is configured to cause the output unit to output images of objects in the one or more image planes along with corresponding object attribute information.

16. The image information display system of claim 14, wherein the object attribute information further includes:
a name of an anatomical part and/or of a portion of the anatomical part;
a clinical characterization of the object;
or both.

17. The image information display system of claim 16, wherein the object attribute information further includes:
object or organ size; a mechanical characteristic; perfusion; hernodynamics; contrast agent dosage uptake; water diffusion rate; contrast agent dosage uptake; organ kinematics and/or dynamics; any combination of the above.

18. An image data segmentation method, comprising:
receiving source image data descriptive of volumetrics objects which were imaged in one or more imaging planes using one or more imaging modalities;
receiving at least one 3D virtual object model that is representable in a plurality of segmentation editing planes and which is associated with non-rigid deformation properties;
displaying a source image of the source image data;
selecting a cross-sectional view of the 3D virtual object model such that the selected cross-sectional view displays a deformable virtual model contour in a segmentation editing plane that corresponds to an imaging plane of the displayed source image; and
displaying the deformable virtual model contour in overlay with the displayed source image.

19. The method of claim 18, further comprising:
actionably engaging the virtual model contour to impart displacement of one or more control points of the virtual model contour for changing display of a current position and/or orientation of the virtual model contour to an updated position and/or orientation; and
displaying the virtual model contour at the updated position and/or orientation.

20. The method of claim 19, wherein the displaying of the object virtual model contour is executed in real-time.
